(19) United States
(12) Patent Application Publication
(10) Pub. No.: US 2002/0123100 A1
(43) Pub. Date: Sep. 5, 2002

(54) Binary BAC vector and uses thereof

(76) Inventors: Maureen R. Hanson, Ithaca, NY (US); Carol Hamilton, Apax, NC (US)

Correspondence Address: NIXON PEABODY LLP, ATTENTION: DAVID RESNICK, 101 FEDERAL STREET, BOSTON, MA, 02110, US

(21) Appl. No.: 09/976,687

(22) Filed: Oct. 12, 2001

Related U.S. Application Data
(60) Provisional application No. 60/241,688, filed on Oct. 19, 2000.

(51) Int. Cl. C12P 21/02; C12N 15/74; C12N 1/21

(52) U.S. Cl. 435/69.1; 435/471; 435/488; 435/252.33

(57) ABSTRACT

The present invention provides a method for transferring and expressing heterologous DNA in a non-plant host cell. The vector used in this method includes a backbone having a first origin of replication capable of maintaining heterologous DNA as a single copy in an Escherichia coli host cell. The vector further includes a unique restriction endonuclease cleavage site for insertion of heterologous DNA, and left and right Agrobacterium T-DNA border sequences flanking the unique restriction endonuclease cleavage site. In certain host cells, the T-DNA border sequences allow introduction of heterologous DNA located between the left and right T-DNA border sequences into a host cell. In preferred embodiments, the vector includes a second origin of replication capable of maintaining heterologous DNA as a single copy in a host cell such as Agrobacterium species or other prokaryotic cells.

### CROSS-REFERENCE TO RELATED APPLICATION

This Utility Application is based on Provisional Application 60/241,688, filed Oct. 19, 2000, the content of which is relied upon and incorporated herein by reference in its entirety, and benefit priority under 35 U.S.C. §119(e) is hereby claimed.

### GOVERNMENT SUPPORT

The subject matter of this application was made with support from the United States Government (National Science Foundation Plant Science Center Grant No. 175-8300-6550-361).

### FIELD OF THE INVENTION

The present invention relates to methods for transferring and expressing heterologous DNA using a bacterial artificial chromosome (BAC) vector and the binary (BIN) vector.

### BACKGROUND OF THE INVENTION

Throughout this application various publications are referenced, many in parenthesis. Full citations for these publications are provided at the end of the Detailed Description. The disclosures of these publications in their entireties are hereby incorporated by reference in this application.

Living organisms exhibit a vast and diverse ability to perform biochemical processes leading to the synthesis of simple and complex molecules. It is often difficult to identify chemicals of interest that are synthesized by living organisms because of the limited quantities of the organism or tissues where the chemical(s) is produced. Quantity may be limited because the organisms cannot be grown in culture or propagated readily outside of their natural environment. Environmental conditions may make it difficult or impossible to collect the organism from nature. A number of standard methods now exist to extract nucleic acids from the environment. DNA copies of RNA or DNA isolated from the environment can be cloned and propagated in bacteria. It is particularly useful to clone large segments of DNA from organisms. In prokaryotes and sometimes eukaryotes, genes in the same biosynthetic or degradatative pathway are sometimes clustered. If a cluster of genes from a wild organism is introduced into a bacterium, the bacterium might express sufficient quantities of gene products from the cloned DNA to carry out the synthesis or degradation of interesting chemicals. However, identification of novel products or biochemical pathways has been hampered by the size of DNA which can be inserted into traditional cloning vectors such as bacteriophage vectors, plasmids or cosmids. Some such commonly used vectors also have the problem that they are not stable in the host, especially if they are present in high copy number.

The vectors typically used for cloning large genomic sequences are: yeast artificial chromosome (YAC), bacterial artificial chromosomes (BAC), P1 or cosmids, usually requiring random subcloning in M13-like vectors. Generation of genomic libraries in YACs allows the cloning of large inserts for long-range physical mapping of complex mammalian genomes (Burke et al., 1987). However, YACs are often unstable and also a difficult source for obtaining pure DNA in sufficient quantities for the preparation of the small-fragment libraries required for DNA sequencing (Osoegawa et al., 1998). Furthermore, many mammalian DNA sequences contain repetitive DNA sequences. Cloning of such repetitive sequences into bacteriophage vectors, plasmids and YAC vectors renders these sequences unstable (Schalkwyk et al., 1995). This results in gaps in physical genomic maps and precludes the use of these vectors as a means of propagating repetitive DNA. Furthermore, Sinden et al., (1991) point to the structural instability of plasmids containing indirect repeats. As with direct repeats, this study shows that there is correlation between the size of the indirect repeat and the degree of structural instability.

It would be desirable to have a method for transferring and expressing large segments of heterologous DNA in a variety of host cells including prokaryotic and non-plant eukaryotic cells.

Hamilton (PCT WO 96/21725; U.S. Pat. No. 5,733,744; C. Hamilton, Gene, 1997; the contents of which are incorporated herein by reference) discloses a vector, referred to as the BIBAC vector for Agrobacterium-mediated plant transformation and formation of genomic libraries. We describe here that the BIBAC vector containing heterologous DNA can be used to transform both prokaryotic and non-plant eukaryotic hosts, including yeast and fungi, and that the heterologous DNA can sometimes be expressed in these hosts. These hosts containing the BIBAC incorporating heterologous DNA can therefore be used in screens for genes for novel biosynthetic or degradative pathways and for production or degradation of various compounds.

### SUMMARY OF INVENTION

The present invention provides a method for transferring and expressing heterologous DNA in a non-plant host cell. The vector used in this method, called BIBAC vector, includes a backbone having a first origin of replication capable of maintaining heterologous DNA as a single copy in an Escherichia coli host cell. The vector further includes a unique restriction endonuclease cleavage site for insertion of heterologous DNA, and left and right Agrobacterium T-DNA border sequences flanking the unique restriction endonuclease cleavage site. In certain host cells, the T-DNA border sequences allow introduction of heterologous DNA located between the left and right T-DNA border sequences into a host cell. In preferred embodiments, the vector includes a second origin of replication capable of maintaining heterologous DNA as a single copy in a host cell such as Agrobacterium species or other prokaryotic cells.

The method of the present invention allows for the construction of genomic libraries with large DNA inserts. The ability to transfer and express large segments of DNA increases the likelihood of cloning a cluster of genes that comprises an entire pathway.

Individual clones can be directly introduced into non-plant host cells by transformation. As used herein, “transformation” or “transforming” means introducing the DNA into the host cell by any appropriate means known in the art. For example, electroporation, calcium phosphate, triparental mating, particle bombardment. Transformation further includes Agrobacterium-mediated transformation.

Non-plant host cells include for example, prokaryotic organisms, filamentous fungi, yeast, insect, and mammalian host cells.

The method of the present invention, by providing for transfer and expression of large segments of DNA in diverse non-plant host cells, advantageously allows one to screen the clones, e.g., a genomic library, for expression of the desired gene product or products in a variety of conditions. For example, using the method of the present invention, a genomic library can be screened for the expression of a desired gene product, e.g. an antibiotic. If the gene product is found not to be expressed in E. coli, the genomic library can then be expressed in another prokaryotic host cell, for example, Agrobacterium tumefaciens. Since Agrobacterium, as a soil bacterium is adapted for growth at lower temperatures than E. coli, it may be better suited for expression of certain gene products e.g., enzymes from microorganisms that prefer to grow in temperatures lower than E. coli. Additionally, the genomic library can further be screened for expression of a desired gene product in a eukaryotic host cell, such as yeast, filamentous fungi and mammalian host cells. Different host species will allow for the synthesis or degradation of different products as a result of differences in their physiological makeup. For example different species have different biochemical pathways. These features underscore the importance of the BIBAC vector in “gene prospecting” i.e. discovery, expression, and production of novel pathways. As well as its use in identifying DNA which encodes genes that results in the production or degradation of important compounds, the BIBAC vector can potentially be used for expression of the DNA for production of the useful compounds in commercial quantities.

### BRIEF DESCRIPTION OF THE DRAWINGS

These and other features and advantages of this invention will be evident from the following detailed description of preferred embodiments when read in conjunction with the accompanying drawings in which:

FIG. 1 shows the map of the BIBAC vector; and

FIG. 2 shows the strategy for construction of the BIBAC vector.

### DETAILED DESCRIPTION

The present invention relates to a method for transferring heterologous DNA into a non-plant host cell. The vector used in this method, designated BIBAC (binary bacterial artificial chromosome vector), includes a backbone having an origin of replication that is capable of maintaining heterologous DNA as a single copy in a bacterial host cell. As used throughout this application, unless otherwise indicated, maintenance as a single copy refers to a non-replicating cell, i.e. a cell not undergoing cell division; during cell division, the copy per cell increases to nearly two complete copies per cell. In certain preferred embodiments the vector includes a second origin of replication capable of maintaining the heterologous DNA as a single copy in an Agrobacterium tumefaciens host cell and in related species. Other origins of replication can be included depending on the desired host cell, if it is desirable to maintain the BIBAC in the host cell rather than transferring the T-DNA into the host's DNA.

The vector also includes a unique restriction endonuclease cleavage site for the insertion of heterologous DNA. The presence of only one cleavage site for a particular restriction endonuclease within the DNA sequence encoding the vector is the presence of a “unique” restriction endonuclease cleavage site. The particular restriction endonuclease will, therefore, only cleave the DNA at that one location or “unique site”.

Heterologous DNA refers to DNA not normally present in the particular host cell transformed by the vector. Heterologous DNA can be obtained from different sources such as prokaryotes; different species such as mammalian, reptile, bird, amphibian, fish etc.; yeast; parasites; environmental samples which may be man-made or natural and may contain mixtures of prokaryotic and eukaryotic organisms; or even spliced genomes from a plurality of organisms. Heterologous DNA can further include libraries of DNA, including genomic libraries.

The vector also includes left and right Agrobacterium T-DNA border sequences flanking the unique restriction endonuclease cleavage site (Peralta and Ream 1985). These border sequences allow the introduction of heterologous DNA located between the left and right T-DNA border sequences into a host cell when using Agrobacterium-mediated DNA transfer.

Stable maintenance of high molecular weight DNA in Escherichia coli and Agrobacterium tumefaciens is made possible because these high molecular weight DNA sequences are carried on a single copy plasmid. With multiple copies of such large DNA inserts, the plasmid might be unstable and if so, the host cells would not be useful for non-plant host cell transformation. This is especially true in cases where the heterologous DNA may encode proteins that are themselves somewhat toxic to the host cell, or in which the heterologous genes' products are toxic, causing selection pressure for loss of the plasmid. Keasling (1999) points out that single-copy plasmids can have the advantage of controlled gene expression and low metabolic burden on the host, but that few such vectors are currently available.

In one embodiment of the present invention, in a vector, designated the BIBAC vector, the unique restriction endonuclease cleavage site is a BamHI cleavage site. This cleavage site is located between the Agrobacterium tumefaciens T-DNA border sequences, as is a selection marker for incorporation of heterologous DNA into the vector (the sacB gene). The BamHI cleavage site and the sacB gene are located such that when heterologous DNA is inserted into the BamHI site the sacB gene is inactivated. The BIBAC vector includes the F origin of replication from Escherichia coli for maintaining the heterologous DNA as a single copy in Escherichia coli (Low 1972), and the Ri origin of replication from Agrobacterium rhizogenes for maintaining the heterologous DNA as a single copy in Agrobacterium tumefaciens and related species. The left and right T-DNA border sequences in the BIBAC vector are derived from the TL-DNA of the octopine plasmid pTiA6.

The BIBAC vector also includes a selection marker for introduction of the heterologous DNA into bacterial cells such as Escherichia coli and Agrobacterium tumefaciens. The bacterial selection marker comprises the kanamycin resistance gene. The original BIBAC vector included a selection marker for introduction of the heterologous DNA into a plant cell. In some non-plant host species, appropriate virulence genes may result in transfer of the DNA between the T-DNA border sequences in host chromosome or extrachromosal elements. In cases where T-DNA transfer is desired, the selection marker must be located between the left and right T-DNA border sequences. The selectable marker must be chosen based on the particular host cell. Markers resulting in complementation of auxotrophic mutations are commonly used in yeast; a kanamycin resistance gene conferring resistance to G418 is commonly used in other eukaryotes (see further below).

The BIBAC vector also includes an origin of conjugal transfer (the oriT origin from plasmid RK2). This origin allows the transfer of heterologous DNA from an Escherichia coli host cell directly to an Agrobacterium tumefaciens host cell and cells of related species by bacterial conjugation.

The vector designated BIBAC has been deposited as the plasmid designated pCH23 in the Escherichia coli strain designated DH10B (pCH23) pursuant to, and in satisfaction of, the requirements of the Budapest Treaty on the International Recognition of the Deposit of Microorganisms for the Purposes of Patent Procedure, with the American Type Culture Collection (ATCC), 12301 Parklawn Drive, Rockville, Md. 20852 under ATCC Accession No. 69743.

The vector is used in the method of the present invention to introduce heterologous DNA into a host cell. Accordingly, the vector preferably includes heterologous DNA inserted into the unique restriction endonuclease cleavage site. DNA is inserted into the vector using standard cloning procedures readily known in the art. This generally involves the use of restriction enzymes (in the case of BIBAC, BamHI) and DNA ligases, as described by Maniatis et al., Molecular Cloning: A Laboratory Manual, Cold Spring Harbor Laboratory Press, Cold Spring Harbor, N.Y. (1982). The vector can then be used to transform a non-plant host cell, such as prokaryotic host cells, e.g. Escherichia coli, Agrobacterium species, Rhizobium species, other related bacterial species, yeast, filamentous fungi, insects and/or a mammalian host cell.

An alternative to the standard method of isolating genomic DNA from cultured organisms is to use amplification methods on DNA extracted from nature. Methods have been established to perform polymerase chain reaction (PCR) and obtain large segments of amplified DNA. Commercial kits are available that can result in amplification of DNAs of length up to 35 kb (see www.stratagene.com). Such DNAs can be incorporated into the BIBAC for heterologous expression.

Heterologous expression of DNA is often most efficient when promoter sequences from the host organism are used to control expression. Such sequences could be cloned into the BIBAC at appropriate locations within the heterologous DNA. For example, a 56 kb gene cluster responsible for synthesis of the polyketide epothilone (a potential anticancer agent) in the myxobacterium Sorangium cellulosum was cloned into two plasmids for heterologous expression in Streptomyces coelicolor (Tang et al., 2000). This cluster could have been cloned as one segment into the BIBAC, with incorporation of appropriate promoters sequences for expression in a chosen host.

For screening of libraries and DNAs isolated from nature for production or degradation of chemicals and products, it is advantageous to look for such phenotypes without further engineering of the heterologous DNA for gene expression. Such engineering requires time-consuming sequencing and characterization of the heterologous DNA, as described in the preceding paragraph for the epothilone pathway. As an alternative to further engineering, hosts containing the BIBAC can be screened for products synthesized by expression of the heterologous DNA from endogenous promoters that happen to be expressed in the host. It is not uncommon for bacterial promoters from one species to be recognized and the gene expressed in other bacterial species. Such recognition is thought to contribute to horizontal gene transfer between species (Davison, 1999; Kroll et al., 1998; Lawrence, 1999). As an example of natural heterologous expression, a DNA fragment of Agrobacterium tumefaciens conferred the ability to metabolize sucrose on E. coli strains (Schuerman et al., 1997). Heterologous expression is not limited to bacterial species; eukaryotic DNA may sometimes be expressed in bacterial host organisms. For example, it has been shown that plant viral and plant promoters can be expressed in E. coli, Yersinia enterocolitica and Agrobacterium tumefaciens (Lewin et al., 1998).

As an alternative to utilizing recognition of heterologous gene regulatory sequences by endogenous host factors, enhanced expression may be achieved by expressing genes for regulatory factors from the source organism in the BIBAC host. For example, enhanced expression of bacterial genes when the BIBAC is incorporated into yeast may be obtained if the RNA polymerase and/or sigma-like factors from the bacterial source of the heterologous DNA are co-expressed in yeast. Such regulatory factor genes could be incorporated into the BIBAC vector or into a second vector or into the chromosomal DNA of the host organism.

The method of the present invention can be used in a number of applications. One application is screening a genomic library for expression of a desired gene product. In this method, genomic DNA from a donor can be cut with a restriction endonuclease (BamHI in the case of the BIBAC vector). The restriction fragments which represent collectively the entire genome of the donor are then each ligated into a vector (which has been opened by cutting with BamHI in the case of the BIBAC vector). This generates a library in the vector. (See generally, Current Protocols in Molecular Cloning, Ausubel, F. M. et al., eds., Greene Publishing and Wiley Interscience, New York (1989).) Alternatively, DNA can be obtained by performing PCR or other gene amplification methods from DNA purified from natural sources, including DNA of non-culturable species. Donor organisms can be any organism such as prokaryotes, animals, plants, and DNA from unknown non-culturable microorganisms.

For ease in working with the library, the vector is generally maintained in a bacterial host cell. Escherichia coli is a standard bacterial host cell for maintaining such a DNA library. The vector DNA can be introduced into the bacterial host cell by various methods known in the art. These include electroporation, calcium chloride transformation, and transformation by particle bombardment. The transformed bacterial cells can be identified by their ability to grow on various selective agents. Accordingly, bacterial cells that contain the vector can be identified by their resistance to kanamycin. The presence of inserted heterologous DNA is indicated by the ability of the bacteria to grow on high levels of sucrose. The ability to grow on high levels of sucrose is due to the inactivation of another selection marker, the sacB gene. Potential clones containing the desired heterologous DNA can be identified by Southern analysis (Southern 1975) using closely linked molecular markers or heterologous DNA as probe, in situ hybridization assays; or PCR probes for detecting sequences related to polypeptides encoded by the heterologous DNA in the vector; oligolabeling, nick translation, end-labeling or PCR amplification using a labeled nucleotide. Clones (vector containing heterologous DNA) of interest are then used for further experiments. The bacterial host containing the clone can be screened for expression of a desired protein or synthesis or degradation of a chemical by methods well known in the art. For example, using either polyclonal or monoclonal antibodies directed against the desired protein, enzyme-linked immunosorbent assay (ELISA), radioimmunoassay (RIA), fluorescent activated cell sorting (FACS), SDS-PAGE gels, Western blots, etc.

If desired, the vector, or a derivative clone of interest (as described above), can be introduced into another prokaryotic host. This introduction can be accomplished using methods known to those in the art, including electroporation, or particle bombardment. Another method that can be used to introduce the vector into certain species such as Agrobacterium tumefaciens is triparental mating. In a triparental mating, the Escherichia coli containing the vector, a second Escherichia coli containing a helper plasmid, and an Agrobacterium are combined, resulting in introduction of the vector DNA into the Agrobacterium. The Agrobacterium cells are then screened using a selection marker (such as kanamycin resistance in the BIBAC vector), for the presence of the vector DNA therein. Those cells containing the vector DNA are then used for further experiments, and/or screened for expression of a desired gene product.

The vector, or a derivative clone of interest (as described above), can also be introduced into an Agrobacterium-transformable non-plant eukaryotic cell. Agrobacterium-transformable non-plant eukaryotic cells include yeast cells such as Saccharomyces cerevisiae, Kluyveromyces lactis; filamentous fungi such as Aspergillus awamori, Aspergillus niger, Fusarium venenatum, Trichoderma reesei, Colletotrichum gloeosporioides, Neurospora crass; and the mushroom Agaricus bisporos. Certain hosts can be transformed by co-culturing with Agrobacterium tumefaciens strains containing the vector. The methodology is well known in the art (Groot et al., 1998; Gouka et al., 1999). Those cells containing the T-DNA are then screened for the expression of the desired gene product.

Alternatively, the vector, or a derivative clone of interest can also be introduced into a non-plant eukaryotic host cell using methods well known in the art including, for example, electroporation, nuclear particle bombardment etc. Those cells containing the vector DNA are then screened for the desired gene product. If necessary, the vector can be modified using known techniques to facilitate DNA expression in the desired eukaryotic host cells.

As used throughout this application, electroporation is a transformation method in which, generally, a high concentration of vector DNA (containing heterologous DNA) is added to a suspension of host cell protoplasts or bacterial cells and the mixture is shocked with an electrical field of 200 to 600 V/cm. Following electroporation, transformed cells are identified by growth on appropriate medium containing a selective agent (Mozo and Hooykaas, 1991).

As also used throughout this application, particle bombardment (also know as biolistic transformation) of the host cell can be accomplished in one of several ways. The first involves propelling inert or biologically active particles at cells. This technique is disclosed in U.S. Pat. Nos. 4,945,050; 5,036,006; and 5,100,792, all to Sanford et al., which are hereby incorporated by reference. Generally, this procedure involves propelling inert or biologically active particles at the cells under conditions effective to penetrate the outer surface of the cell and to be incorporated within the interior thereof. When inert particles are utilized, the vector can be introduced into the cell by coating the particles with the vector containing the heterologous DNA. Alternatively, the target cell can be surrounded by the vector so that the vector is carried into the cell by the wake of the particle. Biologically active particles (e.g., dried bacterial cells containing the vector and heterologous DNA) can also be propelled into cells.

Thus, the methods of the present invention can be used to transform a number of diverse host cells in a variety of ways. Specifically, heterologous DNA encoding the desired gene product, or a library of DNAs can be inserted into the unique restriction endonuclease cleavage site of the vector, e.g. BIBAC. The vector, containing the heterologous DNA, is used to transform a bacterial host cell e.g., Escherichia coli, or Agrobacterium tumefaciens. The transformed bacterial cells can be screened for expression of the desired gene product. The vector can also then be used to transform non-plant host cells including yeast; prokaryotic; mammalian, reptile, bird, etc. Preferably, the host cell is a yeast or filamentous fungus and the transformation is Agrobacterium-mediated. The cells containing the vector can then be screened for expression of the desired gene product. The introduction of the heterologous DNA into the host cell allows the production of the gene product encoded by the heterologous DNA when the DNA is expressed in the cell.

Alternatively, a desired gene product can be produced in a non-plant eukaryotic cell by transforming a cell directly with the vector (having heterologous DNA encoding the gene product inserted into the unique restriction endonuclease cleavage site), such as electroporation.

With such methods in mind, the method of the present invention can be used to isolate genes, including gene clusters, by their phenotype. There are many examples from prokaryotic organisms and some examples from eukaryotic organisms, in which genes involved in the same biosynthetic pathway are genetically and physically linked (Lawrence, 1999). For example, a 25-kb DNA region from Pseudomonas syringae confers upon E. coli the ability to induce hypersensitive response on tobacco—though normally E. coli would not secrete the appropriate signals to cause this response (Alfano et al., 1997). Enzymes which can catalyze the degradation of nicotine are located on a 160-kb plasmid in Arthrobacter nicotinovorans (Shenk and Decker, 1999).

There are also examples of clusters of genes involved in particular biochemical pathways in plants. Pectin methylesterase genes are clustered in Arabidopsis (Richard et al., 1996), two tomato alcohol dehydrogenase genes are closely linked (Ingersoll et al., 1994), and the Brassica self-incompatibility locus spans several hundred kb and contains a number of linked and co-adapted genes (Boyes et al., 1997).

In another embodiment, varying size segments (small-to-large) of DNAs or cDNAs from an organism is cloned into a host bacterium and then the resulting strains screened for production of a product of interest. The host bacterium can be grown either on standard media or fed potential precursors of useful products. For example, antibiotics, novel pigments, enzymes active in temperature extremes, etc.

As used throughout this application, transformation encompasses either transient or stable transformation. In transient transformation, heterologous DNA is introduced into a host cell without being incorporated into the DNA of the host cell (incorporation being a stable transformation).

### EXAMPLE 1

### Construction of the BIBAC

The library features of the BIBAC are based on the bacterial artificial chromosome (BAC) cloning system described by Shizuya et al. (1992). In addition, the vector is a “state of the art” binary vector for Agrobacterium-mediated plant transformation (Hoekema et al. 1983).

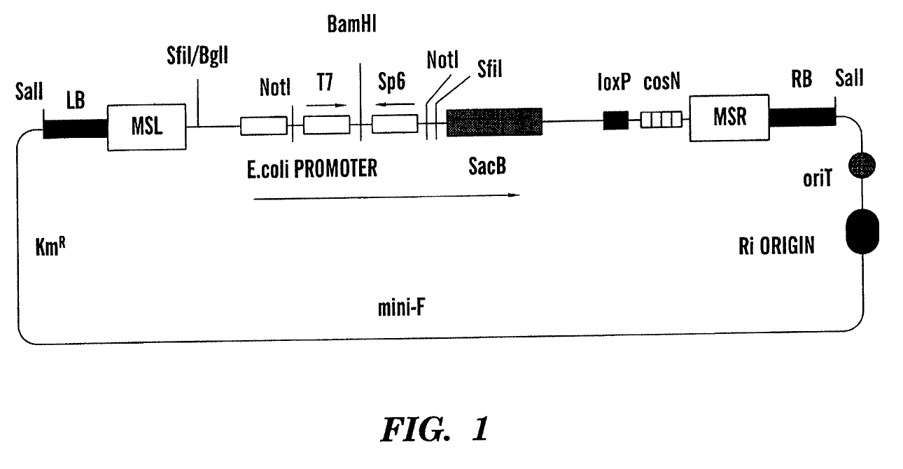

Two major groups of components have been incorporated into the BIBAC. The first group includes those functions that are required in its bacterial hosts: E. coli and A. tumefaciens, and features to help characterize the library. The second group includes those features of the BIBAC which are intended to facilitate Agrobacterium-mediated plant transformation. A map of the BIBAC is shown in FIG. 1.

The backbone of the BIBAC has the minimal region required for F factor replication and maintenance (O'Conner et al. 1989). The λ cosN and P1 loxP sites from pBAC108 are also incorporated into the BIBAC. These sites function as unique restriction sites to anchor one end of the insert and facilitate the analysis of partial digestions with restriction endonuclease(s). The cosN site can be cleaved with the bacteriophage λ terminase (Rackwitz et al. 1985); the loxP site by bacteriophage P1 Cre protein in the presence of the loxP oligonucleotide (Abremski et al. 1983). Restriction maps of the individual clones can be determined by indirect end-labeling and subsequent partial digestion (Rackwitz et al. 1985; Abremski et al. 1983; Kohara et al. 1987).

A marker which provides a positive selection for inserts is incorporated into the BIBAC. This marker is the sacB gene from Bacillus amyloliquifaciens (Tang et al. 1990) which encodes the protein levansucrase. Levansucrase was first identified as a 50 kD protein secreted by Bacillus subtilis following induction by sucrose. The enzyme catalyzes transfructorylation from sucrose to various receptors (Dedonder 1966). The sacB structural gene of B. subtilis was cloned by Gay et al. (1983). Subsequently it was discovered that when 5% sucrose is present in agar media, the production of levansucrase is lethal to E. coli, A. tumefaciens, and Rhizobium meliloti (Gay et al. 1985).

The sacB gene was subcloned from the bacteriophage P1 cloning vector pAd10sacBII (Pierce et al. 1992). (The coding regions of the B. amyloliquifaciens and the B. subtilis sacB genes show 90% identity at the nucleotide level.) This construct has a BamHI cloning site region and a synthetic E. coli promoter upstream of the sacB structural gene. The BamHI cloning site is flanked by T7 and SP6 RNA polymerase promoters which can be used to generate RNA probes for chromosome walking. The Bam HI cloning site is unique to the BIBAC. When a DNA fragment is inserted into the Bam HI site, the sacB gene is inactivated, and the strain is viable when grown on media containing 5% sucrose. The sacB gene was introduced into pBAC108 to generate pCH1. This construct was electroporated into E. coli strain DH10B and the resulting strain was tested for sensitivity to high sucrose by plating a dilution series on standard LB media and on LB containing 5% sucrose. Cells plated on LB containing 5% sucrose showed a plating efficiency of less than 10⁻⁶ compared to cells plated on standard LB media. Thus, plating a potential library on 5% sucrose should yield primarily genomic clones.

The BIBAC has the replication origin region from the Ri plasmid of A. rhizogenes, the causal agent of hairy root disease. The Ti and Ri plasmids belong to different incompatibility groups and can be maintained stably together in one cell (White and Nester 1980; Constantino et al. 1980). This is important because many A. tumefaciens strains used for plant transformation contain a disarmed Ti plasmid as the virulence helper plasmid. The minimal Ri origin region that is incorporated into the BIBAC has been shown to be sufficient for plasmid replication and stable maintenance at 1-2 copies per cell in A. tumefaciens (Jouanin et al. 1985). (The “2” copies per cell is included here to cover the presence of the duplicated DNA during cell replication.) The minimal Ri origin was subcloned from pLJbB11 which was characterized by Jouanin et al. (1985). The oriT cassette and the Ri origin were cloned adjacent to each other in pUC19 (Yanisch-Perron et al. 1985) to create pCH9. This was done so that the two components could be transferred to the vector as a single unit.

The BIBAC has an origin of conjugal transfer (ori T) derived from the wide host range plasmid RK2 of the Inc P group. When all other transfer functions are provided in trans by a helper plasmid, the ori T allows for the conjugal transfer of any covalently linked self replicating DNA (Ditta et al. 1980). The RK2 oriT of the BIBAC is subcloned from a DNA cassette in pNHKan-oriT constructed by Hengen and Iyer (1992). The BIBAC can be transferred to A. tumefaciens by conjugation. The RK2 ori T has been used to effect the conjugal transfer of the chromosomes of E. coli and Rhizobium meliloti (Yakobson and Guiney 1984). Therefore conjugal transfer of high molecular weight DNA from E. coli to Agrobacterium species and other bacterial species is possible. Alternatively, BIBAC clones can be introduced into Agrobacterium species and other bacterial species by electroporation. Mozo and Hooykaas (1991) reported that plasmids as large as 250 kb can be introduced into A. tumefaciens by electroporation.

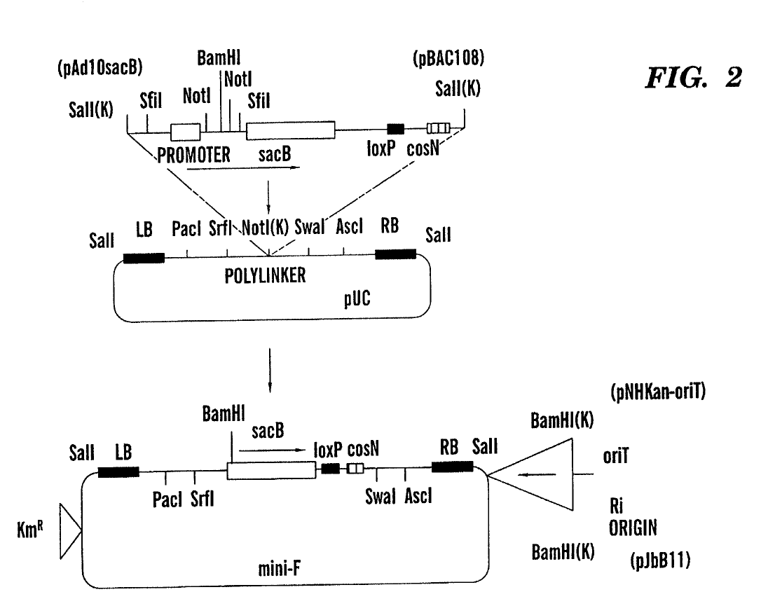

FIG. 2 illustrates the construction of the BIBAC. The right and left border sequences of the BIBAC are derived from the TL-DNA of the octopine plasmid pTiA6. The right border “overdrive” sequences are present (Peralta et al. 1986; van Haaren et al. 1987). The borders in the BIBAC were generated by Pfu polymerase PCR using primers which hybridize to cosmid pVK232 (Knauf and Nester 1982). The plasmid pVK232 contains the entire TL-DNA of TiA6 (de Vos et al. 1981; Barker et al. 1983; Gielen et al. 1984). A synthetic polylinker connects the border sequences, and is designed to provide unique cloning sites adjacent to the right and left border sequences to facilitate the introduction of selectable markers. These are designated MSR (marker site right) and MSL (marker site left) in FIG. 1. Very large segments of DNA between the borders can be transferred into certain host cells (Miranda et al., 1992). The PCR generated borders and the polylinker were initially cloned into pUC 19.

As a result, the right and left borders and the polylinker are carried on a Sal I fragment in pCH7. This was done to facilitate the subsequent cloning of this fragment into the Sal I site of mini-F pMBO131.

The sacB gene was cloned into pCH7 by transforming a pcnB strain of E. coli and incubation of the transformants at 30° C. If a pUC type plasmid is propagated at 30° C., then its copy number is reduced from 80 to 20 copies per cell (Lin-Chao et al. 1992). The pcnB (plasmid copy number) mutation reduces the copy number of ColE1 type plasmids from about 20 copies per cell, to about 1 copy per cell (Lopilato et al. 1986). These conditions were used to obtain the desired construct, pCH10. When the Sal I fragment from pCH10 was introduced into the mini-F to make pCH13, positive clones were easily identified by screening for the sacB marker. The oriT/Ri origin fragment was then introduced into a unique Hpa I site which is 35 nucleotides from the Sal I edge of the mini-F to make pCH16. Preliminary experiments indicated that chloramphenicol is not a useful marker for some strains of Agrobacterium, so a kanamycin resistance gene cassette (Smith and Crouse 1989) was cloned into the Pvu II site of the chloramphenicol gene of the mini-F to complete the backbone of the BIBAC.

The GUS-NPTII construct (Datla et al. 1991) is a bifunctional fusion peptide between E. coli β-glucuronidase (GUS) and neomycin phosphotransferase II (NPTII). This GUS-NPTII construct is carried on an Eco RI-Hind III fragment. The fragment was treated with Klenow and ligated into the Srf I site at the MSL site to create BIBAC1. Though this GUS-NPTII construct is effective as a selectable and screenable marker in plant cells, it will be less useful as a marker in other hosts of the BIBAC, such as microorganisms and animal cells. The GUS-NPTII construct can be excised and replaced with an aminoglycoside 3-phosphotransferase (AGP, kanamycin-resistance) gene which encodes resistance to the drug G418. This drug, in conjunction with an AGP gene under the control of appropriate gene regulatory sequences, has been effective as a selectable marker in diverse types of cells, including yeast, other fungi, insects, and mammalian cells.

If two selectable markers are desired in a BIBAC vector, the promoters and transcription termination sequences of the two constructs should be nonhomologous. This is to prevent any sequence redundancy within the vector which might reduce its stability. In addition, any pairs of selectable markers should be oriented in the BIBAC so that they are not convergently transcribed, as this configuration may impair transgene expression (Jones et al. 1992). When a large segment of DNA is inserted such that the selectable markers are separated by many kb of DNA, this is not likely to be a problem. However, control experiments using the vector alone might be compromised.

Different selectable markers can be incorporated into the BIBAC for maintenance of the plasmid in the host cell of interest. For example, BIBAC3 carries the HYG construct at the MSL. The hygromycin phosphotransferase (HYG) construct provides resistance to hygromycin (Becker, et al. 1992). The HYG construct in BIBAC3 has promoter and terminator regions appropriate for selection in plant cells. These could be replaced with promoters and terminators for other host cells such as fungi or animal cells. Any other selectable marker can be readily introduced at one of the unique polylinker sites. Of course, the selectable marker must not have a Bam HI site, so that the library cloning site will still be unique.

### EXAMPLE 2

### Testing of the BIBAC Vector

A physical map of BIBAC1 has been established, and the vector alone (without any DNA insert) functions as expected. It replicates in E. coli and A. tumefaciens. Tomato and yeast heterologous DNA have been inserted into the BamHI site of the BIBAC vector. Each resulting clone (which includes the vector and the heterologous DNA) was then introduced into Escherichia coli strain DH10B by electroporation. The E. coli strain DH10B has been widely used for construction of genomic libraries, and stability is not expected to be a problem for the majority of BIBAC clones. The DH10B strain contains recal which increases the stability of the inserts, as well as mcrA, mcrB, mcrC, and mrr, which in combination prevent the restriction of DNA which contains methylated cytosine and adenine residues. That is, it should not be a problem to clone even heavily methylated genomic DNA using this strain.

A triparental mating was then performed with the resulting Escherichia coli, an Escherichia coli containing a helper plasmid pRK2073 (which carries resistance to spectinomycin (Leong et al. 1982)), and Agrobacterium tumefaciens. The vector was successfully transferred and was stable in Agrobacterium tumefaciens. These E. coli and Agrobacterium strains carrying the BIBAC with inserts of yeast and plant DNA can be screened by standard methods to detect chemicals and other products produced by the bacteria as a result of expression of the yeast and plant DNA. Alternatively the strains can be screened for the ability to degrade particular chemicals or to utilize particular compounds as carbon or nitrogen sources.

Although preferred embodiments have been depicted and described in detail herein, it will be apparent to those skilled in the relevant art that various modifications, additions, substitutions and the like can be made without departing from the spirit of the invention and these are therefore considered to be within the scope of the invention as defined in the claims which follow.

### REFERENCES

The references cited below and those appearing throughout the application are incorporated herein by reference:

1. Abremski, K. et al. (1983). Studies of the properties of P1 site-specific recombination: evidence for topologically unlinked products following recombination. Cell 32: 1310-1311.

2. Alfano-J-R. Kim-H-S. Delaney-T-P. Collmer-A. (1997). Evidence that the Pseudomonas syringae pv. syringae hrp-linked hrmA gene encodes an Avr-like protein that acts in an hrp-dependent manner within tobacco cells. Molecular Plant-Microbe Interactions. 10: 580-588.

3. Barker, R. F. et al. (1983). Nucleotide sequence of the T-DNA region from the Agrobacterium tumefaciens octopine Ti plasmid pTi15955. Plant Mol. Biol. 2: 335-350.

4. Becker, D. et al. (1992). New plant binary vectors with selectable markers located proximal to the left T-DNA border. Plant Mol. Biol. 20:1195-1197.

5. Boyes-D-C. Nasrallah-M-E. Vrebalov-J. Nasrallah-J-B., (1997). The self-incompatibility (S) haplotypes of Brassica contain highly divergent and rearranged sequences of Ancient origin. Plant Cell 9:237-247.

6. Burke, D. et al. (1987). Cloning of large segments of exogenous DNA into yeast by means of artificial chromosome vectors. Science 236:806-812.

7. Constantino, P. et al. (1980). Tumor formation and rhizogenicity of Agrobacterium rhizogenes carrying Ti plasmids. Gene 11:79-87.

8. Datla, R. S. S. et al. (1991). A bifunctional fusion between β-glucuronidase and neomycin phosphotransferase: a broad-spectrum marker enzyme for plants. Gene 101:239-246.

9. Davison, J. (1999). Genetic exchange between bacteria in the environment. Plasmid 42:73-91.

10. Dedonder, R. (1966). Levansucrase from Bacillus subtilis. Meth. Enzymol. 8:500-505.

11. de Vos, G. et al. (1981). Restriction endonuclease mapping of the octopine tumor inducing plasmid pTiAch5 of Agrobacterium tumefaciens. Plasmid 6:249-253.

12. Ditta, G. et al. (1980). Broad host range DNA cloning system for gram-negative bacteria: Construction of a gene bank of Rhizobium meliloti. Proc. Natl. Acad. Sci. USA. 77:7347-7351.

13. Gay, P. et al. (1983). Cloning structural gene sacB, which codes for exoenzyme levansucrase of Bacillus subtilis: Expression of the gene in Escherichia coli. J. Bact. 153:1424-1431.

14. Gay, P. et al. (1985). Positive selection procedure for entrapment of insertion sequence elements in gram-negative bacteria. J. Bact. 164:918-921.

15. Gielen, J. et al. (1984). The complete nucleotide sequence of the TL-DNA of the Agrobacterium tumefaciens plasmid pTiAch5. EMBO J. 3:835-846.

16. Gouka, R. J. et al. (1999). Transformation of Aspergillus awamori by Agrobacterium tumefaciens-mediated homologous recombination. Nat. Biotech. 17:598-601.

17. Groot, M. J. A. et al. (1998). Agrobacterium tumefaciens-mediated transformation of filamentous fungi. Nat. Biotech. 16:839-842.

18. Hamilton, C. M. (1997). A binary-BAC system for plant transformation with highmolecular-weight DNA. Gene 200:107-116.

19. Hengen, P. N., and Iyer, V. N. (1992). DNA cassettes containing the origin of transfer (oriT) of two broad-host-range transfer systems. BioTech. 13:56-62.

20. Hoekema, A. et al. (1983). A binary plant vector strategy based on separation of vir- and T-region of the Agrobacterium tumefaciens Ti-plasmid. Nature 303:179-180.

21. Ingersoll, J. C., Rothenberg, M., Liedl, B. E., Folkerts, K., Garvin, D., Hanson, M. R., Doyle, J. J., and Mutschler, M. A. (1994). A novel anther-expressed adh-homologous gene in Lycopersicon esculentum. Plant Molec. Biol. 26:1875-1891.

22. Jones, J. D. G. et al. (1992). Effective vectors for transformation, expression of heterologous genes, and assaying transposon excision in transgenic plants. Transgenic Research 1:285-297.

23. Jouanin, L. et al. (1985). Localization and restriction maps of the replication origin regions of the plasmids of Agrobacterium rhizogenes strain A4. Mol. Gen. Genet. 201:370-374.

24. Keasling, J. D. (1999). Gene-expression tools for the metabolic engineering of bacteria. Tibtech 17:452-460.

25. Knauf, V. C., and Nester, E. W. (1982). Wide host range cloning vectors: a cosmid clone bank of an Agrobacterium Ti plasmid. Plasmid 8:45-54.

26. Kohara, Y. et al. (1987). The physical map of the whole E. coli chromosome: application of a new strategy for rapid analysis and sorting of a large genomic library. Cell 50:495-508.

27. Kroll et al. (1998). Natural genetic exchange between Haemophilus and Neisseria: intergeneric transfer of chromosomal genes between major human pathogens. Proc. Nat'l. Acad. Sci. (USA) 95:12381-12385.

28. Lawrence, J. (1999). Selfish operons: the evolutionary impact of gene clustering in prokaryotes and eukaryotes. Curr. Opinion in Genetics and Development 9:642-648.

29. Leong, S. A. et al. (1982). Heme biosynthesis in Rhizobium. Identification of a cloned gene coding for S-aminolevulinic acid synthetase from Rhizobium meliloti. J. Biol. Chem. 257:8724-8730.

30. Lewin, A, D. Jacob, B. Freytag, B. Appel. (1998). Gene expression in bacteria directed by plant-specific regulatory sequences. Transgenic research 7:403-411.

31. Lin-Chao, S. et al. (1992). High copy number of the pUC plasmid results from a Rom/Rop-suppressible point mutation in RNAII. Molec. Microbiol. 6:3385-3393.

32. Lopilato, J. et al. (1986). Mutations in a new chromosomal gene of Escherichia coli K-12, pcnB, reduce plasmid copy number of pBR322 and its derivatives. Mol. Gen. Genet. 205:285-290.

33. Low, K. B. (1972). Escherichia coli K-12 F-prime factors old and new. Bacteriol. Rev. 36:587-607.

34. Miranda, A. et al. (1992). Agrobacterium tumefaciens transfers extremely long T-DNAs by a unidirectional mechanism. J. Bact. 174:2288-2297.

35. Mozo, T., and Hooykaas, P. J. J. (1991). Electroporation of megaplasmid into Agrobacterium. Plant Mol. Biol. 16:917-918.

36. O'Conner, M. et al. (1989). Construction of large DNA segments in Escherichia coli. Science 244:1307-1312.

37. Osoegawa et al. (1998). An improved approach for construction of bacterial artificial chromosome libraries, Genomics 52:1-8.

38. Pace, N. R. (1996). A molecular view of microbial diversity and the biosphere. Science 276:734-740.

39. Peralta, E. G., and Ream, L. W. (1985). T-DNA border sequence required for crown gall tumorigenesis. Proc. Natl. Acad. Sci. USA. 82:5112-5116.

40. Peralta, E. G. et al. (1986). Overdrive, a T-DNA transmission enhancer on the A. tumefaciens tumor-inducing plasmid. EMBO J. 5:1137-1142.

41. Pierce, J. C. et al. (1992). A positive selection vector for cloning high molecular weight DNA by the bacteriophage P1 system: Improved cloning efficacy. Proc. Natl. Acad. Sci. USA. 89:2056-2060.

42. Rackwitz, H. R. et al. (1985). Analysis of cosmids using linearization by phage lambda terminase. Gene 40:259-266.

43. Richard-L. Qin-L-X. Goldberg-R., (1996). Clustered genes within the genome of Arabidopsis thaliana encoding pectin methylesterase-like enzymes. Gene 170:207-211.

44. Saiki, R. K. (1988). Optimization of the polymerase chain reaction. In: Current Communications in Molecular Biology: Polymerase Chain Reaction, H. A. Erlich et al., eds. (New York: Cold Spring Harbor Laboratory Press), pp. 25-30.

45. Schalkwyk, L. C. et al. (1995). Techniques in mammalian genome mapping. Curr. Opin. Biotech. 6:37-43.

46. Schuerman et al. (1997). 3-ketoglycoside-mediated metabolism of sucrose in E. coli as conferred by genes from Agrobacterium tumefaciens. Appl. Microb. Biotech. 47:560-565.

47. Shenk, S. and K. Decker. (1999). Horizontal gene transfer involved in the convergent evolution of the plasmid-encoded enantioselective 6-hydroxynicotine oxidases. J. Mol. Evol. 48(2):178-186.

48. Shizuya, H. et al. (1992). Cloning and stable maintenance of 300-kilobase-pair fragments of human DNA in Escherichia coli using an F-factor-based vector. Proc. Natl. Acad. Sci. USA. 89:8794-8797.

49. Sinden, R. R. et al. (1991). On the deletion of inverted repeated DNA in Escherichia coli: Effects of length, thermal stability, and cruciform formation in vivo. Genetics, 129:991-1005.

50. Smith, M. L., and Crouse, G. F. (1989). Construction of linker-scanning mutations using a kanamycin-resistance cassette with multiple symmetric restriction sites. Gene 84:159-164.

51. Southern, E. M. (1975). Detection of specific sequences among DNA fragments separated by gel electrophoresis. J. Mol. Biol. 98:503-517.

52. Tang, L. B. et al. (1990). Isolation and characterization of levansucrase-encoding gene from Bacillus amyloliquefaciens. Gene 96:89-93.

53. Tang, L., S. Shah, L. Chung, J. Camey, L. Katz, C. Khosla, B. Julien. (2000). Cloning and heterologous expression of the epothilone gene cluster. Science 287: 640-642.

54. van Haaren, M. J. J. et al. (1987). Overdrive is a T-region transfer enhancer which stimulates T-strand production in A. tumefaciens. Nucleic Acids Res. 15:8983-8997.

55. White, F. F., and Nester, E. W. (1980). Relationship of plasmids responsible for hairy root and crown gall tumorigenicity. J. Bact. 144:710-720.

56. Yakobson, E. A., and Guiney, D. G. J. (1984). Conjugal transfer of bacterial chromosomes mediated by the RK2 plasmid transfer origin cloned into transposon Tn5. J. Bact. 160:451-453.

57. Yanisch-Perron et al. (1985). Gene. 33:103-119.

58. Zabeau, M., and Vos, P. (1992): Selective restriction fragment amplification: a general method for DNA fingerprinting. (Assigned to Keygene N.V.) European Patent Application No. EP 91402542, published Sep. 24, 1991. Netherlands Patent No. 0 534 858 A1, issued Mar. 31, 1993.

What is claimed is:

1. A method of introducing heterologous DNA into a non-plant host cell thereby producing a gene product in said cell, said method comprising:
a) inserting heterologous DNA encoding said gene product into a unique restriction endonuclease cleavage site of a vector, said vector comprising:
i) a backbone which includes a first origin of replication capable of maintaining heterologous DNA as a single copy in Escherichia coli host cell, and which further includes a second origin of replication capable of maintaining heterologous DNA as a single copy in an Agrobacterium tumefaciens host cell;
ii) a unique restriction endonuclease cleavage site for insertion of heterologous DNA; and
iii) left and right Agrobacterium T-DNA border sequences flanking said unique restriction endonuclease cleavage site, said left and right T-DNA border sequences allowing introduction of heterologous DNA located between left and right T-DNA border sequences into a non-plant cell;
b) transforming a non-plant cell so as to introduce said heterologous DNA into said cell; and
c) expressing said heterologous DNA in said non-plant cell so as to produce the gene product encoded by said heterologous DNA into said cell.

2. The method of claim 1, wherein said host cell is a yeast cell or a filamentous fungus.

3. The method of claim 2, wherein the yeast cell is Saccharomyces cerevisiae or Kluyveromyces lactis.

4. The method of claim 2 wherein the filamentous fungus is from the genus Aspergillus.

5. A method of producing a gene product in a non-plant host cell, said method comprising:
a) inserting heterologous DNA encoding said gene product into a unique restriction endonuclease cleavage site of a vector, said vector comprising:
i) a backbone which includes a first origin of replication capable of maintaining heterologous DNA as a single copy in Escherichia coli host cell;
ii) a unique restriction endonuclease cleavage site for insertion of heterologous DNA; and
iii) left and right Agrobacterium T-DNA border sequences flanking said unique restriction endonuclease cleavage site, said left and right T-DNA border sequences allowing introduction of heterologous DNA located between left and right T-DNA border sequences into a non-plant host cell;
b) introducing the resulting vector, into said non-plant host cell; and
c) expressing said heterologous DNA in said non-plant host cell so as to produce the gene product encoded by said heterologous DNA.

6. The method of claim 5, wherein said vector further includes a second origin of replication capable of maintaining heterologous DNA as a single copy in an Agrobacterium tumefaciens host cell.

7. The method of claim 5, wherein the non-plant host cell is Escherichia coli.

8. The method of claim 5, wherein the non-plant host cell is a non-plant eukaryotic cell.

9. The method of claim 8, wherein the non-plant eukaryotic cell is a yeast cell.

10. The method of claim 8, wherein the non-plant eukaryotic cell is a mammalian cell.

11. The method of claim 1 or 5, wherein the heterologous DNA is obtained from genomic DNA of prokaryotic cells.

12. The method of claim 1 or 5, wherein the heterologous DNA is obtained from genomic DNA of eukaryotic cells.

13. The method of claim 1 or 5, wherein said first origin of replication comprises an F origin from Escherichia coli.

14. The method of claim 1 or 5, wherein said second origin of replication comprises an Ri origin from Agrobacterium rhizogenes.

15. The method of claim 6, wherein first origin of replication comprises an F origin from Escherichia coli and said second origin of replication comprises an Ri origin from Agrobacterium rhizogenes.

16. The method of claim 1 or 5, wherein said unique restriction endonuclease cleavage site comprises a BamHI cleavage site.

17. The method of claim 1 or 5, wherein said left and right T-DNA border sequences are derived from TL-DNA of octopine plasmid pTiA6.

18. The method of claim 1 or 5, further comprising a selection marker for incorporation of heterologous DNA into said vector.

19. The method of claim 1 or 5, wherein said selection marker comprises a sacB gene, and wherein when heterologous DNA is inserted into unique restriction endonuclease cleavage site of said vector, said sacB gene is inactivated.

20. The method of claim 1 or 5, further comprising a selection marker for introduction of said heterologous DNA into Escherichia coli.

21. The method of claim 1 or 5, wherein said selection marker comprises a kanamycin resistance gene.

22. The method of claim 6, further comprising a selection marker for the introduction of said heterologous DNA into Agrobacterium tumefaciens.

21. The method of claim 1 or 5, further comprising a selection marker for introduction of said heterologous DNA into a non-plant eukaryotic cell, said selection marker located between said left and right T-DNA border sequences.

22. The method of claim 1 or 5, wherein said selection marker is located adjacent to said left T-DNA border sequence.

23. The method of claim 21, wherein said kanamycin resistance gene comprises a GUS-NPTII gene.

24. The method of claim 20, wherein said selection marker comprises a hygromycin resistance gene.

25. The method of claim 1 or 5, wherein said backbone further comprises an origin of conjugal transfer.

26. The method of claim 25, wherein said origin of conjugal transfer comprises an oriT origin from plasmid RK2.

27. A non-plant eukaryotic host cell containing a vector, said vector comprising:
a) a backbone which includes a first origin of replication capable of maintaining heterologous DNA as a single copy in Escherichia coli host cell;
b) a unique restriction endonuclease cleavage site for insertion of heterologous DNA; and
c) left and right Agrobacterium T-DNA border sequences flanking said unique restriction endonuclease cleavage site, said left and right T-DNA border sequences allowing introduction of heterologous DNA located between left and right T-DNA border sequences into a non-plant host cell;
d) a heterologous DNA inserted at said unique restriction endonuclease cleavage site; and
e) a second origin of replication capable of maintaining heterologous DNA as a single copy in an Agrobacterium tumefaciens host cell.

28. The non-plant eukaryotic host cell of claim 27, wherein the host cell is a yeast cell.

29. The non-plant eukaryotic host cell of claim 27, wherein the host cell is a mammalian cell.

30. The host cell of claim 27, wherein the heterologous DNA is from a eukaryotic cell.

31. The host cell of claim 27, wherein the heterologous DNA is from a prokaryotic cell.

32. A method of isolating a DNA encoding a desired gene product from a genomic library of DNA comprising:
a) inserting heterologous DNA from a genomic library of DNA into a vector, said vector comprising:
i) a backbone which includes a first origin of replication capable of maintaining heterologous DNA as a single copy in Escherichia coli host cell;
ii) a unique restriction endonuclease cleavage site for insertion of heterologous DNA; and
iii) left and right Agrobacterium T-DNA border sequences flanking said unique restriction endonuclease cleavage site, said left and right T-DNA border sequences allowing introduction of heterologous DNA located between left and right T-DNA border sequences into a non-plant host cell;
b) introducing the resulting vector, into said non-plant host cell; and
c) expressing said heterologous DNA in said non-plant host cell so as to produce the gene product encoded by said heterologous DNA,
d) screening the cultured host cells for those cells that express the desired gene product, and
e) isolating the DNA encoding the desired gene product from those cells that express the desired gene product.

33. The method of claim 32 wherein said vector further includes a second origin of replication capable of maintaining heterologous DNA as a single copy in an Agrobacterium tumefaciens host cell.

34. The method of claim 32, wherein the host cell is Escherichia coli.

35. The method of claim 32, wherein the host cell is a non-plant eukaryotic cell.

36. The method of claim 32, wherein the non-plant eukaryotic host cell is a yeast cell.

37. The method of claim 32, wherein the non-plant eukaryotic host cell is a mammalian cell.

38. The host cell of claim 32, wherein the genomic library is obtained from prokaryotic cells.

39. The host cell of claim 32, wherein the genomic library is obtained from eukaryotic cells.
